(19) United States
(12) Patent Application Publication
(10) Pub. No.: US 2024/0103024 A1
(43) Pub. Date: Mar. 28, 2024

(54) SYSTEM AND METHOD FOR MATCHING OF STAINING INSTRUMENTS AND SLIDES OF HISTOLOGICAL SAMPLES

(71) Applicant: Leica Biosystems Melbourne, Pty Ltd, Mt. Waverly (AU)

(72) Inventors: Sanatkumar KANDRATTI, Mt. Waverly (AU); Stephly M. STEPHEN, Mt. Waverly (AU)

(21) Appl. No.: 18/530,428

(22) Filed: Dec. 6, 2023

Related U.S. Application Data
(63) Continuation of application No. PCT/US22/15103, filed on Feb. 3, 2022.

(30) Foreign Application Priority Data
Jun. 15, 2021 (IN) 202141026636

(51) Int. Cl. G01N 35/00

(52) U.S. Cl. CPC G01N 35/00732; G01N 2035/00831

(57) ABSTRACT

Features are disclosed for managing slides of tissue sample bocks to be mapped to staining instruments by a slide management apparatus. Each staining instrument may be associated with particular features or characteristics identifying capabilities of the staining instrument. The slide management apparatus can generate groups of slides based on the features or characteristics of each staining instrument. Each group of slides may share the same or similar characteristics. Further, the slide management apparatus can dynamically map the groups of slides to the staining instruments. The slide management apparatus may map the groups of slides based on a staining completion time. The slide management apparatus can generate a recommendation for a user based on mapping the groups of slides. For example, the recommendation may include a recommendation to place a first group of slides in a first staining instrument for staining.

### RELATED APPLICATION

Any and all applications for which a foreign or domestic priority claim is identified in the Application Data Sheet as filed with the present application are hereby incorporated by reference under 37 C.F.R. § 1.57. This application claims the benefit of priority of Indian Provisional Patent Application No. 202141026636, filed Jun. 15, 2021, titled SYSTEM AND METHOD FOR MATCHING OF STAINING INSTRUMENTS AND SLIDES OF HISTOLOGICAL SAMPLES, the entire contents of which is hereby incorporated into this document by reference and made a part of this specification for all purposes, for all that it contains.

### TECHNICAL FIELD

This disclosure generally relates to staining instruments, and more particularly to an instrument configured to stain a slide of a histological sample.

### BACKGROUND

A staining instrument can be used to stain a slide of a histological sample. The staining can be used to highlight particular histological features of the slide (to which a slice of the histological sample may be affixed). The slice can be affixed within a casing to generate a slide. The slide can be used by an imaging system to capture an image of a desired scene. Thereafter, the image can be used for a variety of purposes, including, for example, image analysis. For example, an imaging system can capture an image and perform image analysis on the image to determine particular characteristics or metrics of the image. Examples of imaging systems include, but are not limited to, cameras, scanners, microscopes, mobile devices, tablets, laptops, and/or wearable electronics.

### SUMMARY

One aspect of the present disclosure is a slide management apparatus. The slide management apparatus can include a a first staining instrument configured to stain a slide of a tissue sample to generate a stained slide. The slide management apparatus can further include a transceiver and a computing device including a hardware processor. The hardware processor may receive slide data that identifies a plurality of slides of one or more tissue samples. The hardware processor may further identify the first staining instrument from a plurality of staining instruments. The hardware processor may further access one or more compatibility rules associated with the first staining instrument. Each of the one or more compatibility rules may relate to a characteristic of the first staining instrument. The hardware processor may further group the plurality of slides using the one or more compatibility rules to generate one or more groups of slides. Each of the one or more groups of slides may include a group of compatible slides. The hardware processor may further map a first group of slides of the one or more groups of slides to the first staining instrument. The hardware processor may further cause a transceiver to transmit information indicative of the mapping of the first group of slides to the first staining instrument.

In another aspect of the present disclosure, the hardware processor may further cause the first staining instrument to stain the first group of slides in response to receiving a user acceptance of the information indicative of the mapping of the first group of slides to the first staining instrument.

In another aspect of the present disclosure, each group of compatible slides may share same or similar characteristics.

In another aspect of the present disclosure, each group of compatible slides may include slides sharing a range of similarity. The range of similarity may be defined for a particular staining instrument of the plurality of staining instruments.

In another aspect of the present disclosure, a first slide of the first group of slides of the one or more groups of slides may be associated with a first characteristic value for a characteristic and a second slide of a second group of slides of the one or more group of slides may be associated with a second characteristic value for the characteristic. The first characteristic value and the second characteristic value may be different characteristic values.

In another aspect of the present disclosure, the one or more compatibility rules may include a staining check, a protocol check, or a slide step check.

In another aspect of the present disclosure, the one or more compatibility rules may include a staining method, a staining mode, a slide coverage, a protocol count, a protocol type, a step count, a preferred kit, a fan speed, a fan duration, a dispense profile, a heat application order, or a mixed reagent.

In another aspect of the present disclosure, the hardware processor may further determine a staining completion time for each of the one or more groups of slides based on a staining instrument of the plurality of staining instruments mapped to a particular group of slides. The hardware processor may further map the first group of slides to the first staining instrument based on the staining completion time.

In another aspect of the present disclosure, the information indicative of the mapping of the first group of slides to the first staining instrument may identify an optimized assignment for the plurality of slides.

In another aspect of the present disclosure, to identify the first staining instrument from the plurality of staining instruments, the hardware processor may receive one or more of a rack availability or a reagent rack availability for the first staining instrument.

In another aspect of the present disclosure, to identify the first staining instrument from the plurality of staining instruments, the hardware processor may identify an available staining instrument of the plurality of staining instruments. The available staining instrument may include the first staining instrument.

In another aspect of the present disclosure, the plurality of staining instruments include a second staining instrument. The hardware processor may map a second group of slides of the one or more groups of slides to the second staining instrument.

In another aspect of the present disclosure, the information indicative of the mapping of the first group of slides to the first staining instrument identifies racks of the first staining instrument for placement of the first group of slides.

In another aspect of the present disclosure, to receive the slide data, the hardware processor may receive the slide data from a scanning device, a user computing device, or any other computing device.

In another aspect of the present disclosure, the slide of the tissue sample may be associated with at least one of: a tag, an RFID tag, a Bluetooth tag, an identifier, a barcode, a label, a marker, or a stamp.

In another aspect of the present disclosure, the slide management apparatus may include a coverslipper to archive the slide of the tissue sample.

In another aspect of the present disclosure, the tissue sample may include a paraffin embedded tissue sample, a OCT-embedded tissue sample, a frozen tissue sample, or a fresh tissue sample.

In another aspect of the present disclosure, the slide management apparatus may include a microtome that slices the tissue sample to generate the slide of the tissue sample.

In another aspect of the present disclosure, the hardware processor may further cause the transceiver to transmit the information indicative of the mapping of the first group of slides to the first staining instrument to a user computing device associated with a user.

In another aspect of the present disclosure, the hardware processor may further cause display of the information indicative of the mapping of the first group of slides to the first staining instrument via a user computing device associated with a user.

In another aspect of the present disclosure, the hardware processor may further receive, via the transceiver, a response to the information indicative of the mapping of the first group of slides to the first staining instrument and update a mapping of the first group of slides to the first staining instrument. To update the mapping of the first groups of slides to the first staining instrument, the hardware processor may map the first group of slides of the one or more groups of slides to a second staining instrument of the plurality of staining instruments based on the response to the information indicative of the mapping of the first group of slides to the first staining instrument.

In another aspect of the present disclosure, the hardware processor may further cause the transceiver to transmit the information indicative of the mapping of the first group of slides to the first staining instrument via an application programming interface.

The foregoing summary is illustrative only and is not intended to be limiting. Other aspects, features, and advantages of the systems, devices, and methods and/or other subject matter described in this application will become apparent in the teachings set forth below. The summary is provided to introduce a selection of some of the concepts of this disclosure. The summary is not intended to identify key or essential features of any subject matter described herein.

### BRIEF DESCRIPTION OF THE DRAWINGS

Various examples are depicted in the accompanying drawings for illustrative purposes, and should in no way be interpreted as limiting the scope of the examples. Various features of different disclosed examples can be combined to form additional examples, which are part of this disclosure.

FIG. 1 depicts a schematic diagram of an example networked environment according to some embodiments.

FIG. 2 depicts an example workflow for generating image data from a tissue sample block according to some embodiments.

FIG. 3A illustrates an example tissue block sample according to some embodiments.

FIG. 3B illustrates an example tissue block sample and an example tissue slice sample affixed to a slide according to some embodiments.

FIG. 4 depicts an imaging system for capturing images of the histological samples according to some embodiments.

FIG. 5A depicts a schematic diagram of an example networked environment for outputting a recommendation, according to some embodiments.

FIG. 5B provides a schematic diagram of software communicatively coupled to a group of staining devices.

FIG. 6 depicts a schematic diagram of a slide management system for determining compatibility of slides, according to some embodiments.

FIG. 7 depicts an example interface for requesting the generation of a recommendation, according to some embodiments.

FIG. 8A is a flowchart of an example routine for mapping slides to staining instruments according to some embodiments.

FIG. 8B is a flowchart of an example workflow that utilizes the technique shown in FIG. 8A.

FIG. 8C provides further details regarding certain aspects of the workflow shown in FIG. 8B.

FIG. 9 shows an example computing device that may be used to implement aspects of the present disclosure.

### DETAILED DESCRIPTION

Generally described, the present disclosure relates to a slide management system (e.g., a slide analysis system) that can receive data identifying one or more slides of one or more histological samples (e.g., tissue blocks) and data identifying one or more staining instruments (e.g., instruments for staining the one or more slides). The slide management system can dynamically map or otherwise allocate the one or more slides to the one or more staining instruments based on compatibility rules prior to the loading of the one or more slides into the one or more staining instruments. Based on the dynamic mapping, the slide management system can generate a compatibility report and/or plan (e.g., a recommendation) that identifies the mapping between the one or more slides and the one or more staining instruments. Based on the compatibility report, the slide management system may recommend placement of a subset of the slides in a first staining instrument and placement of another subset of the slides in a second staining instrument.

In order to determine staining instruments that are compatible with particular slides, the slide management system can implement a slide management process or algorithm that analyzes the slides and the staining instruments. As described herein, the use of such a slide management process can increase the efficiency of the staining process and decrease a number of errors and/or delays during the staining process. For example, the slide management process may dynamically map the slides to the staining instruments prior to the loading of the slides into the staining instruments thereby increasing the efficiency of the staining process and reducing the risk that the slides are incompatible. Further, the number of slides and the number of staining instruments can be large and it may be inefficient to determine slide compatibility after loading the slides into the staining instruments. For example, where the system includes 30 slides and 10 staining instruments, it may be inefficient to divide the slides between the staining instruments and determine the compatibility after loading the slides into the staining instruments. The use of the slide management process can greatly reduce the time required to implement a staining process. Further, the slide management process can increase the efficiency of the staining instruments and the efficient utilization of the staining instruments. Additionally, the slide management process may be desirable where the slides correspond to a plurality of protocols. For example, some users may customize and use different protocols for different slides. The different protocols may or may not be compatible with particular staining instruments. Determining if staining instruments are compatible with particular slides after loading the slides into the staining instruments may be an arduous and inefficient process. The slide management process may improve on this process by dynamically mapping the slides, based on the corresponding protocols to staining instruments, prior to the slides being uploaded to the staining instruments. Further, the slide management process may receive updated slides and/or updated slide definitions, and, in real time, update the dynamic mapping of the slides to the staining instruments based on the updated slides and/or the updated slide definitions. Therefore, the slide management process may increase the efficiency of the staining process.

As used herein, the term “staining instrument” may refer to any electronic or component(s) capable of performing a histological staining. For example, a “staining instrument” may comprise a slide stainer, an automated stainer, a compact stainer, etc. As used herein, the term “imaging system” may refer to any electronic device or component(s) capable of performing an imaging process. For example, an “imaging system” may comprise a scanner, a camera, etc. In some embodiments, the imaging system may not perform the imaging and, instead, may receive the image data and perform image analysis on the image data.

As described herein, a staining instrument can be used to perform slide staining on received slides (each slide can correspond to one or more slices of histological samples). The staining instrument can receive a plurality of slides corresponding to a plurality of histological samples. Each slide and each histological sample can be associated with a particular tissue block and/or a section of a particular tissue block. Further, each slide and each histological sample may be collected in order to identify particular characteristics and/or particular information. For example, a first slide may be collected in order to identify a first protein within a corresponding tissue block and a second slide may be collected in order to identify a second protein within a corresponding tissue block. As each slide may be collected for different reasons (e.g., to identify different features or characteristics), the staining process may vary for each slide. For example, a first staining process may be a routine staining process, a second staining process may be a specialized staining process using specific dyes to identify particular features, etc. As each staining instrument may not be capable of performing each possible staining process, it is important to ensure that a particular staining instrument is capable of performing the desired staining process for a given slide. As multiple slides may be uploaded at one time to a staining instrument, if the staining instrument determines that it is unable to perform a particular staining process for a particular slide, the staining process for the multiple slides may be delayed with the particular slide is identified and replaced.

Each histological sample and each slide of the histological sample can be associated with an identifier and, based on these identifiers, the staining instrument can determine the staining process associated with the slide and verify that the staining instrument is capable of performing the staining process for the slide. In order to ensure that compatible slides are uploaded to the staining instruments, the slide management system can perform compatibility checks to confirm that the slides recommended for a particular staining instrument are compatible with the staining instrument (e.g., the staining instrument is capable of performing the associated staining processes).

In many conventional cases, implementing a generic staining process and/or a generic staining instrument may not provide satisfactory results in particular circumstances or for particular users. Such generic staining instruments may receive a series of slides (e.g., by a user uploading the series of slides) and, after receiving the series of slides, the generic staining instruments may perform compatibility checks to determine whether the staining instrument is compatible with the slides. For example, the generic staining instruments may scan the slides uploaded to the staining instrument (e.g., by scanning an identifier of the slides) to identify staining processes for each slide. Such a generic staining instrument may cause non-compatible slides to be erroneously matched and uploaded to the staining instrument based upon a user input. For example, the user may place 10 slides in the staining instrument and the staining instrument may not be capable of processing 2 of the 10 slides. As the generic staining instrument performs the compatibility check after the slides are uploaded, the user may have to remove the slides and determine which slides are incompatible with the staining instrument which may be an inefficient process. As the slides correspond to histological samples (e.g., tissue blocks), it can be crucial to execute the staining process efficiently. Uploading slides to the staining instrument that are incompatible with the staining instrument may cause significant delays and may result in a delay of medical diagnosis or treatment. Such a delay of medical diagnosis or treatment can lead to additional adverse consequences. Additionally, by requiring manual user input in order to determine slides that are incompatible with the staining instrument, the traditional staining process can lead to additional performance issues. For example, the staining process for a generic staining instrument may be slow, inefficient, and non-effective. Conventional staining instruments may therefore be inadequate in the aforementioned situations.

As staining systems proliferate, the demand for faster and more efficient staining has also increased. The present disclosure provides a system for staining slides of histological samples with significant advantages over prior implementations. The present disclosure provides systems and methods that enable an increase in the efficiency of the staining process, relative to traditional staining systems, without significantly affecting speed or accuracy. These advantages are provided by the embodiments discussed herein, and specifically by the implementation of a slide management system that dynamically maps one or more slides to one or more staining instruments. Further, the use of the slide management system enables the system to update the dynamic mapping in real time in response to identifying additional slides and/or additional staining instruments. The use of the slide management system may further allow the determination of a desired mapping of slides to staining instrument (e.g., the mapping of slides to the staining instruments that will be the most efficient or result in the quickest staining process), thereby increasing the efficiency of the staining process according to the above methods.

Some aspects of this disclosure relate to dynamically mapping slides of histological samples to staining instruments. The slide management apparatus or system described herein can provide improved accuracy for staining by using a module or system that can identify the mapping of slides to instruments with the highest efficiency. The use of such a module is able to provide increased efficiency without significantly impacting computation speeds provided by conventional staining systems. Further, each staining instrument may be associated with one or more compatibility rules identifying characteristics of the staining instrument. For example, the compatibility rules may identify capabilities of the staining instrument (e.g., a range of capabilities of the staining instrument). The slide management apparatus can utilize the compatibility rules in order to generate groups of slides and then map each group of slides to a corresponding staining instrument for staining based on a slide management process. The slide management system can further generate a recommendation for a particular staining instrument and/or a rack of a staining instrument for each slide.

Each slide within a group of slides may share certain characteristics. For example, each slide may have characteristics within a certain range of values based on the compatibility rules. In some embodiments, the slides in a group of slides may have different values for a particular characteristic. The different values may be within a particular range of similarity as defined by a compatibility rule. For example, a compatibility rule may identify that a fan duration between 0 and 10 seconds is compatible and characteristics for fan duration with a value between 0 and 10 seconds may be considered. This compatibility check may be performed for each characteristic of a particular slide. Further, the compatibility checks may be performed between the particular slide and each other slide of the group of slides. The compatibility checks may include a staining check, a protocol check, and/or a slide step check. Further, the compatibility checks may include monitoring or checking a staining method, a staining mode, a slide coverage, a protocol count, a protocol type, a step count, a preferred kit, a fan speed, a fan duration, a dispense profile, a heat application order, and/or a mixed reagent.

Other aspects of this disclosure relate to identifiers that can provide identifying information for a particular slide. For example, the identifiers can identify a source of the histological sample, a patient associated with the histological sample, a unique code, a staining process, a staining protocol, or any other identifying information. Based on the identifiers, the slide management system can determine if a particular slide is compatible with a particular staining instrument using the compatibility rules. For example, the slide management system can determine that a first slide associated with a first identifier is compatible with the staining instrument and a second slide associated with a second identifier is not compatible with the staining instrument. In some embodiments, the slide management system may not use identifiers to identify the slides. For example, the slide management system may prompt a user to provide or identify characteristics for a particular slide.

Other aspects of this disclosure relate to a determination, by the slide management system, of a time optimized dynamic mapping. The slide management system may determine a staining completion time for each group of slides. Based on the staining completion time, for each group of slides, the slide management system can dynamically map the groups of slides to the staining instruments such that the dynamic mapping identifies the time optimized mapping of the groups of slides to the staining instruments. For example, the dynamic mapping may identify the mapping of the groups of slides to the staining instruments that will result in the quickest completion of the staining process for each of the groups of slides as compared to other potential mappings.

Other aspects of this disclosure relate to update the dynamic mapping of groups of slides to staining instruments. For example, the slide management system may update the dynamic mapping of groups of slides to staining instruments in response to determining that a new slide has been added to the slides for dynamic mapping and/or that details about slides or staining instruments have been modified. Therefore, the slide management system can generate a dynamic mapping for the new slide and/or update a prior dynamic mapping for particular slides.

In the following description, various examples will be described. For purposes of explanation, specific configurations and details are set forth in order to provide a thorough understanding of the examples. However, it will also be apparent to one skilled in the art that the examples may be practiced without the specific details. Furthermore, well-known features may be omitted or simplified in order not to obscure the examples being described.

### System Overview

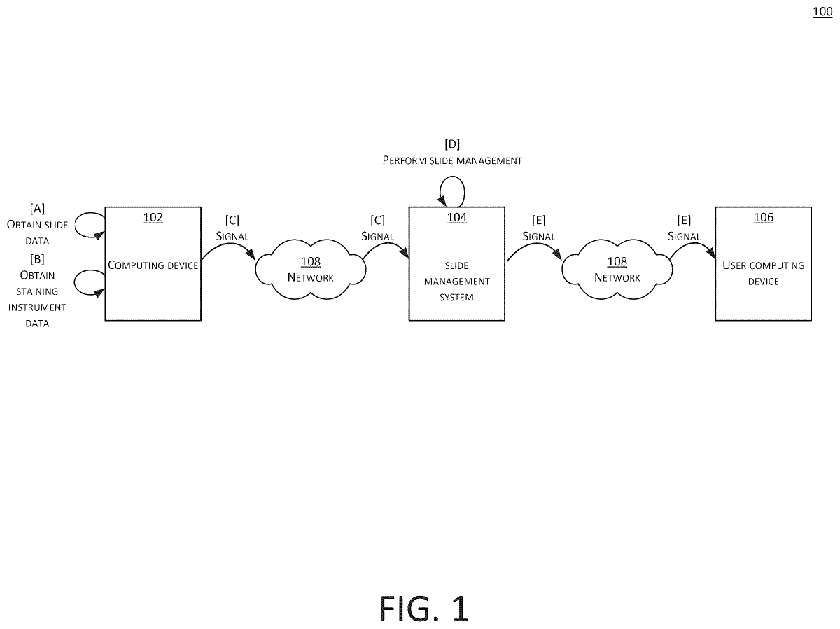

FIG. 1 illustrates an example environment 100 in which a user and/or a system may implement a slide management system 104 according to some embodiments. The slide management system 104 may perform slide management on slides of histological samples. The slide management system 104 can perform slide management in order to determine an optimized mapping of the slides of histological samples to staining instruments. Based on the optimized mapping, the slide management system 104 can generate a recommendation for a user.

The slide management system 104 may perform the slide management using a slide management module (not shown in FIG. 1). The slide management system 104 may receive slide data and staining instrument data from a computing device 102. In some embodiments, the slide management system 104 may separately receive compatibility rules from the computing device 102. The slide management system 104 may transmit the recommendation to a user computing device 106 for processing. Although some examples herein refer to a specific type of device as being the computing device 102, the slide management system 104, or the user computing device 106, the examples are illustrative only and are not intended to be limiting, required, or exhaustive. The slide management system 104 may be any type of computing device (e.g., a server, a node, a router, a network host, etc.). Further, the computing device 102 may be any type of computing device (e.g., a mobile device, a laptop, etc.). In some embodiments, the computing device 102 may include a microtome to generate the slides of the histological sample. Further, the user computing device 106 may be any type of computing device (e.g., a mobile device, a laptop, etc.).

The computing device 102 may obtain (capture, generate, etc.) slide data and staining instrument data for analysis. The computing device 102 may include one or more of a processor and/or memory. The computing device 102 may further receive a user interaction. The user interaction may be a request to implement the slide management process. Based on the user interaction, the computing device 102 may capture the slide data and the staining instrument data. In some embodiments, the slide management process may be an automated process that runs automatically. For example, the slide management process may be performed for a user and/or a user may opt-in to automatic performance of the slide management process. In some embodiments, the computing device 102 may capture the slide data and the staining instrument data periodically (e.g., every 10, 20, or 30 minutes). In other embodiments, the computing device 102 may determine that a new slide has been generated and/or that a new slide (or staining instrument) has been added to a group of slides for the slide management process and, based on this determination, obtain the slide data and/or the staining instrument data as updated.

The computing device 102 may further receive slide data and/or the staining instrument data from additional computing devices. For example, the computing device 102 may be a node that routes slide data and/or the staining instrument data from other computing devices (e.g., the staining instruments, a microtome) to the slide management system 104. In some embodiments, the computing device 102 may be located within the slide management system 104. For example, the computing device 102 may be a component of the slide management system 104. In other embodiments, the computing device 102 and the slide management system 104 may be connected (e.g., via a wireless or wired connection). For example, the computing device 102 and the slide management system 104 may communicate over a network 108. Further, the computing device 102 and the slide management system 104 may communicate over a wired connection. In one embodiment, the slide management system 104 may include a docking station that enables the computing device 102 to dock with the slide management system 104.

The slide management system 104 may include various components for providing the features described herein. In some embodiments, the slide management system 104 may include one or more slide management modules to perform the slide management of the slide data and the staining instrument data received from the computing device 102. The slide management modules may perform one or more dynamic mapping processes for the slide data and the staining instrument data.

The slide management system 104 may be connected to the user computing device 106. The slide management system 104 may be connected (via a wireless or wired connection) to the user computing device 106 to provide a recommendation for the dynamic mapping of the slides to the staining instruments. The slide management system 104 may transmit the recommendation to the user computing device 106 via the network 108. In some embodiments, the slide management system 104 and the user computing device 106 may be configured for connection such that the user computing device 106 can engage and disengage with slide management system 104 in order to receive the recommendation. For example, the user computing device 106 may engage with the slide management system 104 upon determining that the slide management system 104 has generated a recommendation for the user computing device 106. Further, a particular user computing device 106 may connect to the slide management system 104 based on the slide management system 104 performing slide management on the slides and the staining instruments that correspond to the particular user computing device 106. For example, a user may be associated with a plurality of histological samples and a plurality of staining instruments. Upon determining, that a particular histological sample is associated with a particular user and a corresponding user computing device 106, the slide management system 104 can transmit a recommendation for mapping slides of the histological samples to particular staining instruments to the particular user computing device 106. In some embodiments, the user computing device 106 may dock with the slide management system 104 in order to receive the recommendation.

In some implementations, the computing device 102, the slide management system 104, and/or the user computing device 106 may be in wireless communication. For example, the computing device 102, the slide management system 104, and/or the user computing device 106 may communicate over a network 108. The network 108 may include any viable communication technology, such as wired and/or wireless modalities and/or technologies. The network may include any combination of Personal Area Networks (“PANs”), Local Area Networks (“LANs”), Campus Area Networks (“CANs”), Metropolitan Area Networks (“MANs”), extranets, intranets, the Internet, short-range wireless communication networks (e.g., ZigBee, Bluetooth, etc.), Wide Area Networks (“WANs”)—both centralized and/or distributed—and/or any combination, permutation, and/or aggregation thereof. The network 108 may include, and/or may or may not have access to and/or from, the internet. The computing device 102 and the slide management system 104 may communicate slide data and staining instrument data, e.g., via one or more communication devices which may include transmitter(s), receiver(s) and/or transceiver(s). For example, the computing device 102 may communicate slide data associated with a histological sample to the slide management system 104 via the network 108 for mapping. The slide management system 104 and the user computing device 106 may communicate a recommendation corresponding to the slide data and the staining instrument data. For example, the slide management system 104 may communicate a recommendation indicating the dynamic mapping of slides to staining instruments to the user computing device 106. In some embodiments, the computing device 102 and the slide management system 104 may communicate via a first network and the slide management system 104 and the user computing device 106 may communicate via a second network. In other embodiments, the computing device 102, the slide management system 104, and the user computing device 106 may communicate over the same network.

With reference to an illustrative embodiment, at [A], the computing device 102 can obtain slide data. In order to obtain the slide data, a microtome may generate one or more slides of a tissue block (e.g., by slicing the tissue block). The tissue block may be a histological sample. For example, the tissue block may be a block of biological tissue that has been removed and prepared for analysis. As will be discussed in further detail below, in order to prepare the tissue block for analysis, various histological techniques may be performed on the tissue block. Further, a user (e.g., the user associated with the user computing device 106) can store (e.g., generate) the slide data identifying one or more staining processes and/or protocols for each slide. The slide data may be accessed by scanning an identifier (e.g., an RFID tag, a barcode, etc.) associated with the slide. The computing device 102 can interact (e.g., scan, capture, record, etc.) with the identifier in order to obtain the slide data. The computing device 102 can obtain the slide data and store the slide data in the computing device 102. The computing device 102 may obtain the slide data based on a user interaction. For example, a user may provide an input through a user interface (e.g., a graphical user interface (“GUI”)) and request that a particular slide be included in a plurality of slides to be dynamically mapped to staining instruments. In some embodiments, the user can provide the slide data as input to the computing device 102. In other embodiments, the user can interact with computing device 102 to cause the computing device 102 to scan the identifier of the slide. In some embodiments, the computing device 102 may scan the identifier of a slide, based on detecting, by the computing device 102, that a slide has been placed in a viewport of the computing device 102. For example, the computing device 102 may determine that a slide has been placed on a viewport of the computing device 102 and, based on this determination, scan the identifier of the slide.

At [B], the computing device 102 can obtain staining instrument data. In some embodiments, separate computing devices can obtain the slide data and the staining instrument data. For example, a first computing device can obtain the slide data and a second computing device can obtain the staining instrument data. In some embodiments, the staining instrument data may include compatibility rules. For example, the staining instrument data may identify the compatibility rules associated with each staining instrument. Further, the staining instrument data may identify a first subset of compatibility rules associated with a first staining instrument and a second subset of compatibility rules associated with a second staining instrument. In other embodiments, the computing device 102 or a separate computing device may separately receive the compatibility rules. In order to obtain the staining instrument data, the computing device 102 can image (e.g., scan, capture, record, etc.) an identifier associated with each staining instrument. The identifier for the staining instruments may be affixed to each staining instrument and the user may utilize the computing device 102 to scan each identifier. The computing device 102 may obtain the staining instrument data based on a user interaction. For example, a user may provide an input through a user interface (e.g., a graphical user interface (“GUI”)) and request that a particular staining instrument be included in a plurality of staining instruments be dynamically mapped to slides. In some embodiments, the user can provide the staining instrument data as input to the computing device 102. In other embodiments, the computing device 102 may identify available staining instruments of a plurality of staining instruments (e.g., staining instruments that are powered, include one or more available racks, and/or are connected to a network). In some embodiments, the computing device 102 may identify a subset of the available staining instruments (e.g., previously used staining instruments, preferred staining instruments, etc.). The computing device 102 can further store data associated with the available staining instruments as the staining instrument data.

At [C], the computing device 102 can transmit a signal to the slide management system 104 representing the captured data (e.g., the staining instrument data and the slide data). The computing device 102 can send the captured data as an electronic signal to the slide management system 104 via the network 108. The signal may include and/or correspond to a representation of the staining instrument data and the slide data. It will be understood that the signal can include and/or correspond to more, less, or different data. For example, the signal may correspond to multiple slides of a tissue block and/or multiple staining instruments. Further, the signal may enable the slide management system 104 to reconstruct the staining instrument data and/or the slide data. In some embodiments, the computing device 102 can transmit a first signal corresponding to the staining instrument data and a second signal corresponding to the slide data. In other embodiments, a first computing device can transmit a signal corresponding to the staining instrument data and a second computing device can transmit a signal corresponding to the slide data.

At [D], the slide management system 104 can perform a slide management process based on the staining instrument data and the slide data provided by the computing device 102. The slide management system 104 may further perform the slide management process based on one or more compatibility rules (e.g., obtained from the staining instrument data or from separately received data). The slide management system 104 may utilize the slide data and the one or more compatibility rules to identify groups of slides. Each group of slides may share the same and/or similar characteristics. The definition of same and/or similar characteristics may be defined using the compatibility rules. For example, a compatibility rule may identify that a first staining instrument is capable of fan speeds between 0 and 10 miles per hour. Slices that are associated with a staining protocol or process requiring fan speeds between 0 and 10 miles per hour may be considered to have a same and/or similar characteristic (e.g., if a first slide requires a fan speed of 2 miles per hour and a second slide requires a fan speed of 9 miles per hour) based on the compatibility rules. In some embodiments, to define a group of slides, each slide of the group may be required to share the same and/or similar characteristics for each compatibility rule associated with a particular staining instrument. It will be understood that a given staining instrument may be associated with any number of compatibility rules. Based on generating the groups of slides, the slide management system 104 can dynamically map each group of slides to a particular staining instrument. The slide management system 104 may generate multiple mappings of the groups of slides to the staining instruments and identify an optimized mapping. The optimized mapping may correspond to the mapping requiring the least amount of time and/or the best use of resources. The slide management system 104 can further generate a recommendation based on the dynamic mapping. The recommendation may recommend the user upload the slides to the staining instruments based on the dynamic mapping.

At [E], the slide management system 104 can transmit a signal to the user computing device 106 representing a recommendation identifying the dynamic mapping of slides to the staining instruments. The slide management system 104 can send the recommendation as an electrical signal to the user computing device 106 via the network 108. The signal may include and/or correspond to a representation of the recommendation. Based on receiving the signal, the user computing device 106 can determine the recommendation. In some embodiments, the slide management system 104 may transmit a series of recommendations corresponding to a group of staining instruments and slides. The slide management system 104 can include, in the recommendation, a recommended action of a user. For example, the recommendation may include a recommendation for the user to upload particular slides to particular staining instruments. In some embodiments, the slide management system 104 may update the recommendation in real time based on identification of additional slides, additional staining instruments, and/or modification to the slide data or the staining instrument data.

### Imaging Prepared Blocks and Prepared Slides

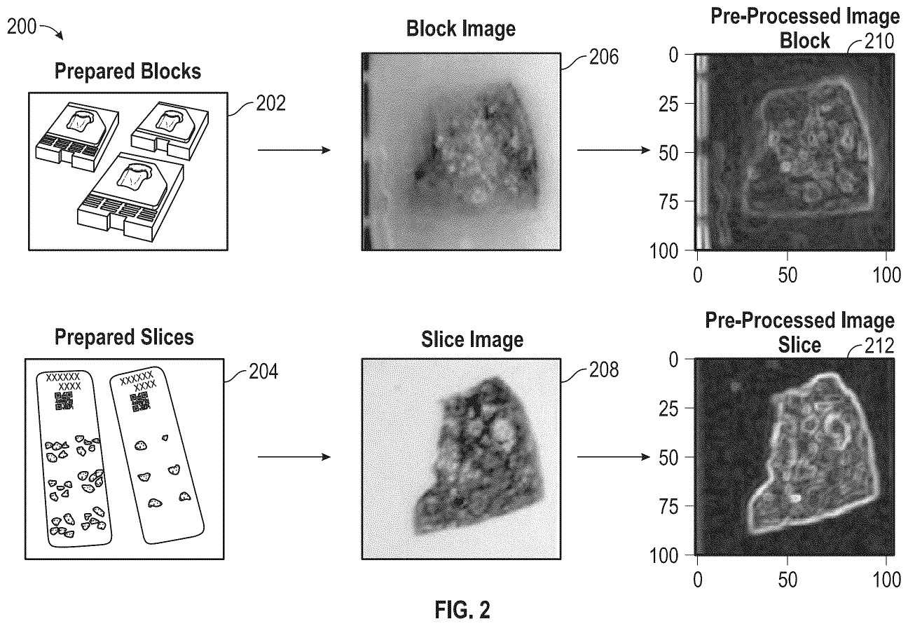

FIG. 2 depicts an example workflow 200 for generating image data from a tissue sample block according to some embodiments. The example workflow 200 illustrates a process for generating prepared blocks and prepared slides including slices from a tissue block and generating pre-processed images based on the prepared blocks and the prepared slides. The example workflow 200 may be implemented by one or more computing devices. For example, the example workflow 200 may be implemented by a microtome, a coverslipper, a staining instrument, and an imaging device. Each computing device may perform a portion of the example workflow. For example, the microtome may cut the tissue block in order to generate one or more slices of the tissue block. The coverslipper may create a first slide for the tissue block and/or a second slide for a slice of the tissue block, the staining instrument may stain each slide, and the imaging device may image each slide.

A tissue block can be obtained from a patient (e.g., a human, an animal, etc.). The tissue block may correspond to a section of tissue from the patient. The tissue block may be surgically removed from the patient for further analysis. For example, the tissue block may be removed in order to determine if the tissue block has certain characteristics (e.g., if the tissue block is cancerous). In order to generate the prepared blocks 202, the tissue block may be prepared using a particular preparation process by a tissue preparer. For example, the tissue block may be preserved and subsequently embedded in a paraffin wax block. Further, the tissue block may be embedded (in a frozen state or a fresh state) in a block. The tissue block may also be embedded using an optimal cutting temperature (“OCT”) compound. The preparation process may include one or more of a paraffin embedding, an OCT-embedding, or any other embedding of the tissue block. In the example of FIG. 2, the tissue block is embedded using paraffin embedding. Further, the tissue block is embedded within a paraffin wax block and mounted on a microscopic slide in order to formulate the prepared block.

The microtome can obtain a slice of the tissue block in order to generate the prepared slides 204. The microtome can use one or more blades to slice the tissue block and generate a slice (e.g., a section) of the tissue block. The microtome can further slice the tissue block to generate a slice with a preferred level of thickness. For example, the slice of the tissue block may be 1 millimeter. The microtome can provide the slice of the tissue block to a coverslipper. The coverslipper can encase the slice of the tissue block in a slide to generate the prepared slides 204. The prepared slides 204 may include the slice mounted in a certain position. Further, in generating the prepared slides 204, a staining instrument may also stain the slice of the tissue block using any staining protocol. Further, the staining instrument may stain the slice of the tissue block in order to highlight certain portions of the prepared slides 204 (e.g., an area of interest). In some embodiments, a computing device may include both the coverslipper and the staining instrument and the slide may be stained as part of the process of generating the slide.

The prepared blocks 202 and the prepared slides 204 may be provided to an imaging device for imaging. In some embodiments, the prepared blocks 202 and the prepared slides 204 may be provided to the same imaging device. In other embodiments, the prepared blocks 202 and the prepared slides 204 are provided to different imaging devices. The imaging device can perform one or more imaging operations on the prepared blocks 202 and the prepared slides 204. In some embodiments, a computing device may include one or more of the tissue preparer, the microtome, the coverslipper, the staining instrument, and/or the imaging device.

The imaging device can capture an image of the prepared block 202 in order to generate the block image 206. The block image 206 may be a representation of the prepared block 202. For example, the block image 206 may be a representation of the prepared block 202 from one direction (e.g., from above). The representation of the prepared block 202 may correspond to the same direction as the prepared slides 204 and/or the slice of the tissue block. For example, if the tissue block is sliced in a cross-sectional manner in order to generate the slice of the tissue block, the block image 206 may correspond to the same cross-sectional view. In order to generate the block image 206, the prepared block 202 may be placed in a cradle of the imaging device and imaged by the imaging device. Further, the block image 206 may include certain characteristics. For example, the block image 206 may be a color image with a particular resolution level, clarity level, zoom level, or any other image characteristics.

The imaging device can capture an image of the prepared slides 204 in order to generate the slide image 208. The imaging device can capture an image of a particular slice of the prepared slides 204. For example, a slide may include any number of slices and the imaging device may capture an image of a particular slice of the slices. The slide image 208 may be a representation of the prepared slides 204. The slide image 208 may correspond to a view of the slice according to how the slice of the tissue block was generated. For example, if the slice of the tissue block was generated via a cross-sectional cut of the tissue block, the slide image 208 may correspond to the same cross-sectional view. In order to generate the slide image 208, the prepared slides 204 may be placed in a cradle of the imaging device (e.g., in a viewer of a microscope) and imaged by the imaging device. Further, the slide image 208 may include certain characteristics. For example, the slide image 208 may be a color image with a particular resolution level, clarity level, zoom level, or any other image characteristics.

The imaging device can process the block image 206 in order to generate a pre-processed image 210 and the slide image 208 in order to generate the pre-processed image 212. The imaging device can perform one or more image operations on the block image 206 and the slide image 208 in order to generate the pre-processed image 210 and the pre-processed image 212. The one or more image operations may include isolating (e.g., focusing on) various features of the pre-processed image 210 and the pre-processed imaged 212. For example, the one or more image operations may include isolating the edges of a slice or a tissue block, isolating areas of interest within a slice or a tissue block, or otherwise modifying (e.g., transforming) the block image 206 and/or the slide image 208. In some embodiments, the imaging device can perform the one or more image operations on one of the block image 206 or the slide image 208. For example, the imaging device may perform the one or more image operations on the block image 206. In other embodiments, the imaging device can perform first image operations on the block image 206 and second image operations on the slide image 208.

### Slicing a Tissue Block

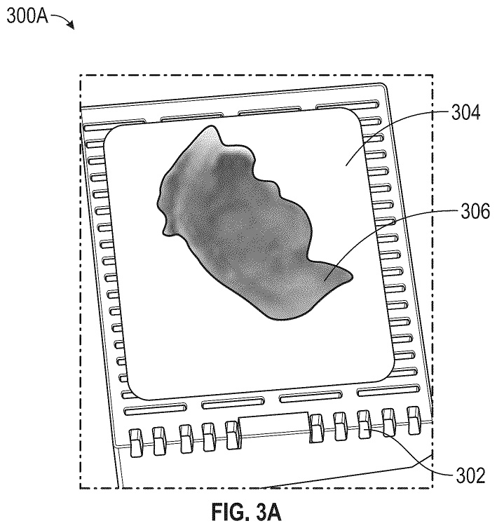

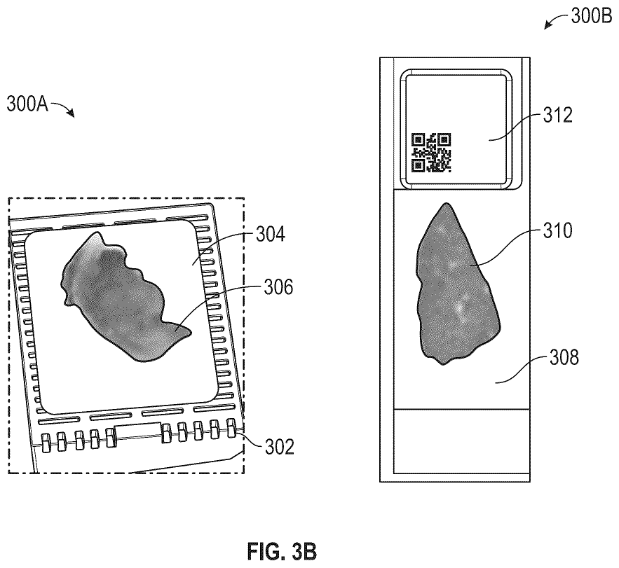

FIG. 3A illustrates an example prepared tissue block 300A according to some embodiments. The prepared tissue block 300A may include a tissue block 306 that is preserved (e.g., chemically preserved, fixed, supported) in a particular manner. In order to generate the prepared tissue block 300A, the tissue block 306 can be placed in a fixing agent (e.g., a liquid fixing agent). For example, the tissue block 306 can be placed in a fixative such as formaldehyde solution. The fixing agent can penetrate the tissue block 306 and preserve the tissue block 306. The tissue block 306 can subsequently be isolated in order to enable further preservation of the tissue block 306. Further, the tissue block 306 can be immersed in one or more solutions (e.g., ethanol solutions) in order to replace water within the tissue block 306 with the one or more solutions. The tissue block 306 can be immersed in one or more intermediate solutions. Further, the tissue block 306 can be immersed in a final solution (e.g., a histological wax). For example, the histological wax may be a purified paraffin wax. After being immersed in a final solution, the tissue block 306 may be formed into a prepared tissue block 300A. For example, the tissue block 306 may be placed into a mould filled with the histological wax. By placing the tissue block in the mould, the tissue block 306 may be moulded (e.g., encased) in the final solution 304. In order to generate the prepared tissue block 300A, the tissue block 306 in the final solution 304 may be placed on a platform 302. Therefore, the prepared tissue block 300A may be generated. It will be understood that the prepared tissue block 300A may be prepared according to any tissue preparation methods.

FIG. 3B illustrates an example prepared tissue block 300A and an example prepared slide 300B with an affixed tissue slice according to some embodiments. The prepared tissue block 300A may include the tissue block 306 encased in a final solution 304 and placed on a platform 302. In order to generate the prepared slide 300B, the prepared tissue block 300A may be sliced by a microtome. The microtome may include one or more blades to slice the prepared tissue block 300A. The microtome may take a cross-sectional slice 310 of the prepared tissue block 300A using the one or more blades. The cross-sectional slice 310 of the prepared tissue block 300A may include a slice 310 (e.g., a section) of the tissue block 306 encased in a slice of the final solution 304. In order to preserve the slice 310 of the tissue block 306, the slice 310 of the tissue block 306 may be modified (e.g., washed) to remove the final solution 304 from the slice 310 of the tissue block 306. For example, the final solution 304 may be rinsed and/or isolated from the slice 310 of the tissue block 306. Further, as will be discussed in further detail below, the slice 310 of the tissue block 306 may be stained by a staining instrument in order to identify particular features of the slice 310. In some embodiments, the slice 310 of the tissue block 306 may not be stained. The slice 310 of the tissue block 306 may subsequently be encased in a slide 308 by a coverslipper to generate the prepared slide 300B. The prepared slide 300B may include an identifier 312 identifying the tissue block 306 that corresponds to the prepared slide 300B. Not shown in FIG. 3B, the prepared tissue block 300A may also include an identifier that identifies the tissue block 306 that corresponds to the prepared tissue block 300A. As the prepared tissue block 300A and the prepared slide 300B correspond to the same tissue block 306, the identifier of the prepared tissue block 300A and the identifier 312 of the prepared slide 300B may identify the same tissue block 306.

### Imaging Devices

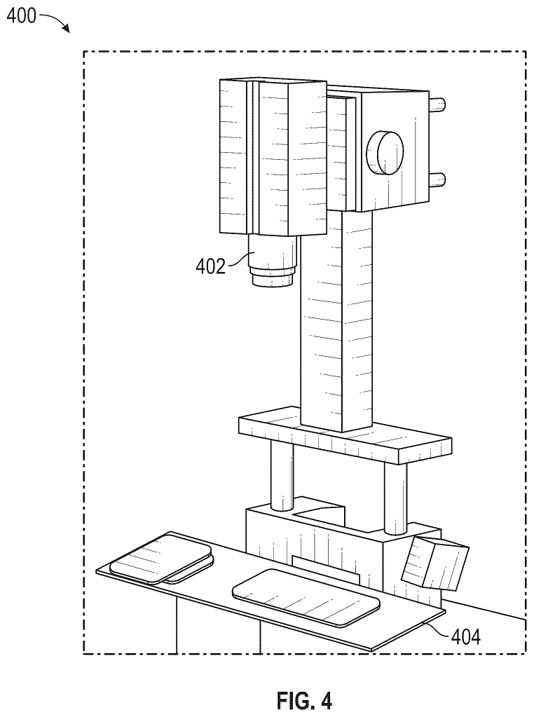

FIG. 4 shows an example imaging device 400, according to one embodiment. The imaging device 400 can include an imaging apparatus 402 (e.g., a lens and an image sensor) and a platform 404. The imaging device 400 can receive a prepared tissue block and/or a prepared tissue slide with an affixed tissue slice via the platform 404. Further, the imaging device can use the imaging apparatus 402 to capture image data corresponding to the prepared block and/or the prepared slide. The imaging device 400 can be one or more of a camera, a scanner, a medical imaging device, etc. Further, the imaging device 400 can use imaging technologies such as microscopy imaging technologies. For example, the imaging technologies may include brightfield microscopy, darkfield microscopy, phase contrast microscopy, differential interference contrast microscopy, fluorescence microscopy, polarizing microscopy, kohler illumination, oil immersion microscopy, light microscopy, immunofluorescence microscopy, chromogenic in situ hybridization microscopy, in situ hybridization microscopy, fluorescence in situ hybridization microscopy, or any other type of imaging technology. For example, the imaging device can be a darkfield microscope, a brightfield microscope, a fluorescent microscope, etc.

The imaging device 400 may receive one or more of the prepared tissue block and/or the prepared slide and capture corresponding image data. In some embodiments, the imaging device 400 may capture image data corresponding to a plurality of prepared tissue slides and/or a plurality of prepared tissue blocks. The imaging device 400 may further capture, through the lens of the imaging apparatus 402, using the image sensor of the imaging apparatus 402, a representation of a prepared tissue slide and/or a prepared tissue block as placed on the platform. Therefore, the imaging device 400 can capture image data associated with a particular slice of a histological sample or a particular histological sample.

### Slide Management System

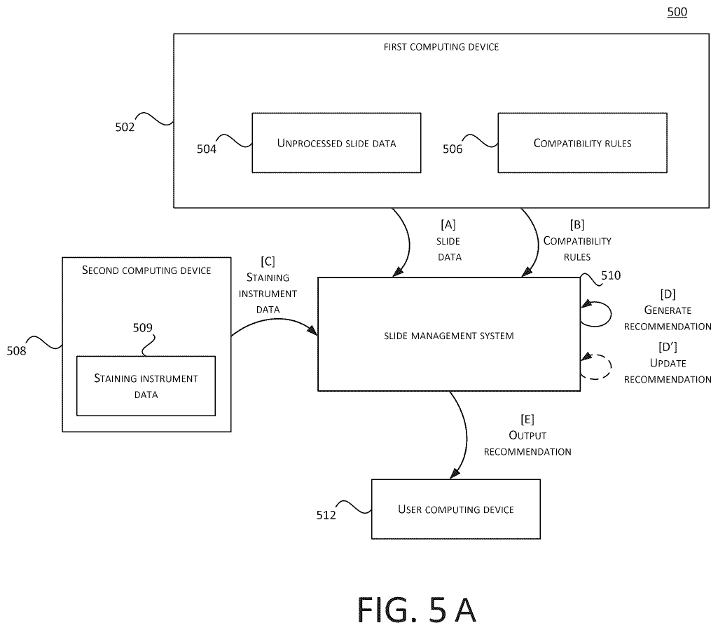

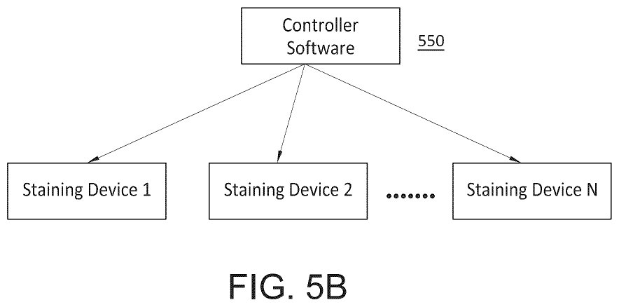

FIG. 5A illustrates an example environment 500 in which a user and/or a system may implement a slide management system 510 according to some embodiments. The slide management system 510, sometimes referred to as a slide compatibility planner, may perform slide management by dynamically mapping, allocating, or assigning slides to staining instruments. Based on the optimized mapping, the slide management system 510 can generate and output a recommendation for a user.

The slide management system 510 may receive slide data 504 (e.g., unprocessed slide data) and compatibility rules 506 from a first computing device 502. The first computing device 502 may further store the slide data 504 and/or the compatibility rules 506 for access by the slide management system 510. In some embodiments, the slide management system 510 may receive or retrieve compatibility rules from a source or location other than the first computing device 502. The slide management system 510 may receive staining instrument data 509 from a second computing device 508. The second computing device 508 may further store the staining instrument data 509 for access by the slide management system 510. In some embodiments, the slide management system 510 may receive one or more of the slide data 504, the compatibility rules 506, and/or the staining instrument data 509 from the same computing device. The slide management system 510 may further utilize one or more of the slide data 504, the compatibility rules 506, and/or the staining instrument data 509 in order to generate a recommendation for a user. Further, the slide management system 510 can output the recommendation to a user computing device 512 for processing. Although some examples herein refer to a specific type of device as being the first computing device 502, the second computing device 508, the slide management system 510, or the user computing device 512, the examples are illustrative only and are not intended to be limiting, required, or exhaustive. The first computing device 502, the second computing device 508, the slide management system 510, or the user computing device 512 may be any type of computing devices.

With reference to an illustrative embodiment, at [A], the slide management system 510 can obtain slide data 504 from the first computing device 502. The slide data 504 may be unprocessed slide data (e.g., the slide data may correspond to slides that have not been stained by a staining instrument). The first computing device 502 may periodically or aperiodically provide the slide data 504 to the slide management system 510. The slide data 504 may be provided to the slide management system 510 as slides are generated by a microtome. For example, a microtome can generate the one or more slides of a tissue block by slicing the tissue block and, as each slide is generated, the first computing device 502 can update the slide data 504. Further, the first computing device 502 can provide updated slide data to the slide management system 510. For example, the first computing device 502 may identify (e.g., based on user input, based on scanning previously unprocessed slides) slide data that was not previously provided to the slide management system 510. In response to this determination, the first computing device 502 can provide the slide data 504 to the slide management system 510 in order to update slide data stored by the slide management system 510.

At [B], the slide management system 510 can obtain compatibility rules 506 from the first computing device 502. The compatibility rules 506 may be one or more rules associated with particular staining instruments and/or particular slides. The compatibility rules 506 may further identify a value or a range of values for particular characteristics of slides. The compatibility rules 506 may define that slides are compatible (e.g., matching) if the characteristics of the slides match the value or are within the range of values identified by a particular compatibility rule. In some embodiments, each compatibility rule may correspond to multiple characteristics and may identify a value for each characteristic. For example, a particular compatibility rule may correspond to a particular staining instrument and may correspond to each characteristic of the staining instrument. In other embodiments, multiple compatibility rules may correspond to the same staining instrument and each compatibility may correspond to a particular characteristic associated with the staining instrument. The compatibility rules 506 may be provided to the slide management system 510 as slide data 504 and/or staining instrument data 509 is obtained. In some embodiments, a separate computing device may obtain the compatibility rules 506 and provide the compatibility rules 506 to the slide management system 510. For example, the second computing device 508 may provide the staining instrument data 509 and the compatibility rules 506 to the slide management system 510.

At [C], the slide management system 510 can obtain staining instrument data 509 from the second computing device 508. The staining instrument data 509 may identify available staining instruments, available racks within the available staining instruments, and/or available rack positions within the available staining instruments. For example, the staining instrument data 509 may identify available slide or staining racks and/or available reagent racks, wherein the slide racks correspond to where the slides are inserted for staining, and wherein the reagent rack corresponds to where reagents required for staining are inserted provided. The second computing device 508 may periodically or aperiodically provide the staining instrument data 509 to the slide management system 510. The staining instrument data 509 may be provided to the slide management system 510 as staining instruments are detected by the second computing device 508. Further, the staining instrument data 509 may be provided to the slide management system 510 as the availability of the staining instruments changes. For example, the second computing device 508 may determine that a staining process for a first group of slides has been completed by the staining instrument and/or the first group of slides has been removed from the staining instrument. In response to this determination, the second computing device 508 can provide staining instrument data 509 identifying that at least the positions within the straining instrument previously occupied by the first group of slides are available. Further, the second computing device 508 can provide updated staining instrument data to the slide management system 510. For example, the second computing device 508 may identify (e.g., based on user input, based on scanning previously unidentified staining instruments) staining instrument data that was not previously provided to the slide management system 510. In response to this determination, the second computing device 508 can provide the staining instrument data 509 to the slide management system 510 in order to update staining instrument data stored by the slide management system 510.

At [D], the slide management system 510 can generate a recommendation. The recommendation may be based on the slide data 504, the compatibility rules 506, and the staining instrument data 509. In order to generate the recommendation, the slide management system 510 may utilize the slide data 504 and the compatibility rules 506 to generate groups of slides. Further, the slide management system 510 can utilize the staining instrument data 509 to map the groups of slides to available positions within the staining instruments based on the staining instrument data 509. The slide management system 510 may utilize the mapping to generate a recommendation for the user. The recommendation can include a recommendation to upload the slides into the identified staining instruments based on the mapping. At [D′], the slide management system 510 can update a previously generated recommendation. The slide management system 510 may determine to update the previously generated recommendation based on receiving updated staining instrument data, updated slide data, or updated compatibility rules. In response to receiving the updated information, the slide management system 510 can perform an updated mapping of the slides to the staining instruments. Further, the slide management system 510 can update the recommendation based on the updated mapping of the slides to the staining instruments.

At [E], the slide management system 510 can output the recommendation to the user computing device 512. The recommendation may identify the mapping by the slide management system 510 and may request physical implementation of the mapping. In some embodiments, the slide management system 510 may provide the recommendation to an automated device (e.g., a robot, an electronic device, etc.) and may cause the automated device to upload the slides to the staining instruments based on the mapping.

In one or more aspects of the present disclosure, the slide management system 510 checks the compatibility of the slides and facilitates effective utilization of lab instruments and resources to load the slides, e.g., into one or more staining devices or systems. These solutions may be implemented with controller software or the like. For example, the controller software may be a Bond controller software. It will be understood that the controller software may be implanted using any controller software. Further, the controller software may be implemented on the first computing device 502, the second computing device 508, the slide management system 510, and/or the user computing device 512.

With reference to FIG. 5B, the controller software 550 may be in operative communications with a first staining device 1, a second staining device 2, etc., up to, for example, N staining devices.

The controller software may be configured to manage the slides, reagents, protocol information, and instrument connections/communications. In related aspects, the controller software may be configured to control the messages to and from the instruments to perform advance staining of slides. In further related aspects, the controller software may be configured to manage multiple staining devices in a lab environment, such as, for example, ranging from 5 to 30 staining devices.

The staining device or instrument generally refers to staining device hardware subsystems which are configured to receive instruction(s) from the controller software and perform the staining operation on the slides using the appropriate reagents, e.g., loaded by the user. The staining device hardware may have common resources (e.g., heater, fan, etc.) for each of the slide lanes of the staining device.

### Compatibility Checks

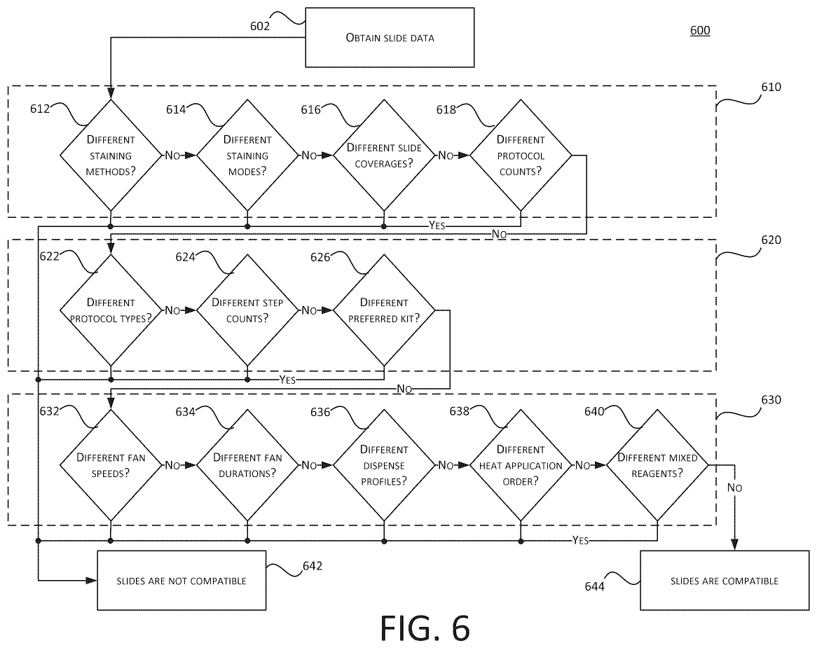

FIG. 6 depicts a schematic diagram 600 of a slide management system for determining compatibility of slides, according to some embodiments.

The slides loaded on to a staining device or instrument should be compatible with each other so that device can process these slides continuously using the device hardware resource effectively. If incompatible slides were loaded onto a given device, then alerts or notifications would be provided to the user, who in turn would have to rearrange the slides among the devices or remove one or more of the slides from the given device.

The schematic diagram 600 identifies a series of compatibility checks to be performed by the slide management system in order to determine if slides are compatible—for example, based on types of staining, protocols used, and/or other parameters like fan speed, fan duration, dispense profiles etc. The slide management system can utilize the output to generate a recommendation for a user. It will be understood that the schematic diagram may include more, less, or different compatibility checks.

The slide management system can perform various types of compatibility checks. In the example of FIG. 6, the schematic diagram 600 includes the following types of checks: staining checks 610, protocol checks 620, and slide step checks 630. Each type of compatibility check may include one or more compatibility checks that are grouped based on the type of compatibility check. It will be understood that the slide management system may perform more, less, or different types of compatibility checks that each may include more, less, or different compatibility checks. It will further be understood that the slide management system may perform

At block 602, the slide management system can obtain slide data. For example, the slide management system may obtain the slide data from a computing device associated with a microtome. The slide data may identify updated slide data, slide data associated with previously unprocessed slides, or any other slide data. The slide management system may then perform the compatibility checks based on the slide data prior to the slides corresponding to the slide data being uploaded into a staining instrument. The compatibility checks may determine are different or the same/similar (e.g., within an acceptable range of difference as defined by the compatibility checks).

Based on obtaining the slide data, the slide management system can compare two or more slides. The slide management system may perform the staining checks 610 on the two or more slides. In a first step of performing the staining checks 610, the slide management system may determine if the two or more slides have different staining methods 612. For example, a first slide may correspond to a first staining method (e.g., immunohistochemistry staining) and a second slide may correspond to a second staining method (e.g., in situ hybridization) as linked or defined by a user. In a second step of performing the staining checks 610, the slide management system may determine if the two or more slides have different staining modes 614. For example, a first slide may correspond to a parallel staining mode and a second slide may correspond to a sequential staining mode. In a third step of performing the staining checks 610, the slide management system may determine if the two or more slides have different slide coverages 616. For example, a first slide may correspond to a 150 millimeter area and a second slide may correspond to a 100 millimeter area. In a fourth step of performing the staining checks 610, the slide management system may determine if the two or more slides have different protocol counts. For example, a first slide may correspond to a 10 protocol count and a second slide may correspond to an 11 protocol count. It will be understood that the staining checks 610 may include more, less or different compatibility checks. It will further be understood that the staining checks 610 may be ordered in any manner. If the stainings are different and/or not within an acceptable range of difference (as defined by the compatibility checks) with regard to any of the staining checks 610, the slide management system may determine the slides are not compatible at block 642.

Based on determining the two or more slides pass the staining checks 610, the slide management system may perform the protocol checks 620. In a first step of performing the protocol checks 620, the slide management system may determine if the two or more slides have different protocol types 622. For example, a first slide may correspond to a first protocol type (e.g., type A) and a second slide may correspond to a second protocol type (e.g., type B) as linked or defined by a user. In a second step of performing the protocol checks 620, the slide management system may determine if the two or more slides have different step counts 624. For example, a first slide may correspond to a first amount of step counts (e.g., the staining process includes 15 steps) and a second slide may correspond to a second amount of step counts (e.g., the staining process includes 20 steps). In a third step of performing the protocol checks 620, the slide management system may determine if the two or more slides have different preferred kit 626. For example, a first slide may correspond to a preferred kit for the staining process and a second slide may not correspond to a preferred kit for the staining process. It will be understood that the protocol checks 620 may include more, less or different compatibility checks. It will further be understood that the protocol checks 620 may be ordered in any manner. If the protocols are different and/or not within an acceptable range of difference (as defined by the compatibility checks) with regard to any of the protocol checks 620, the slide management system may determine the two or more slides are not compatible at block 642.

Based on determining the two or more slides pass the staining checks 610 and the protocol checks 620, the slide management system may perform the slide step checks 630. In a first step of performing the slide step checks 630, the slide management system may determine if the two or more slides have different fan speeds 632. For example, a first slide may require a first fan speed (e.g., 5 miles per hour) and a second slide may require a second fan speed (e.g., 10 miles per hour) as linked or defined by a user. In a second step of performing the slide step checks 630, the slide management system may determine if the two or more slides have different fan durations 634. For example, a first slide may require a first fan duration (e.g., 10 seconds) and a second slide may require a second fan duration (e.g., 20 seconds). In a third step of performing the slide step checks 630, the slide management system may determine if the two or more slides have different dispense profiles 636. For example, a first slide may correspond to a first dispense profile and a second slide may correspond to a second dispense profile. In a fourth step of performing the slide step checks 630, the slide management system may determine if the two or more slides have different heat application orders 638. For example, a first slide may correspond to a first heat application order and a second slide may correspond to a second heat application order. In a fifth step of performing the slide step checks 630, the slide management system may determine if the two or more slides have different mixed reagents 640. For example, a first slide may correspond to a first reagent and a second slide may correspond to a second reagent. It will be understood that the slide step checks 630 may include more, less or different compatibility checks. It will further be understood that the slide step checks 630 may be ordered in any manner. If the slide steps are different and/or not within an acceptable range of difference (as defined by the compatibility checks) with regard to any of the slide step checks 630, the slide management system may determine the two or more slides are not compatible at block 642. If the two or more slides satisfy (e.g., pass) the staining checks 610, the protocol checks 620, and the slide step checks 630 (and any other applicable staining checks), the slide management system may determine the two or more slides are compatible at block 644 with respect to a particular staining instrument as identified by the compatibility checks. Therefore, the slide management system may utilize the schematic diagram 600 to determine if two or more slides are compatible with respect to a particular staining instrument.

In one illustrative example, a case is created for a patient and the doctor is asking for two types of staining on the tissue slide: in situ hybridization (ISH) and immunohistochemistry (IHC). Two slides may have to be created where two different types of protocols have to be used. If one were to try to load these slides into the staining device or instrument, the system or component(s) thereof should not allow a user to run these slides as single batch because of the compatibility issues. The user would have to load these slides separately, and reagents would have to be loaded according to the protocols into the respective instruments.

With some systems, this compatibility check happens after loading the slides into the staining instrument, and the incompatibility is known only after the slides are loaded on the staining instrument (e.g., the Bond-III/Bond-Max instruments (Clinical and Rx)). The technique shown in FIG. 6 prevents such inefficiencies and allows for determining optimized groupings of slides into the staining devices.

### Interface

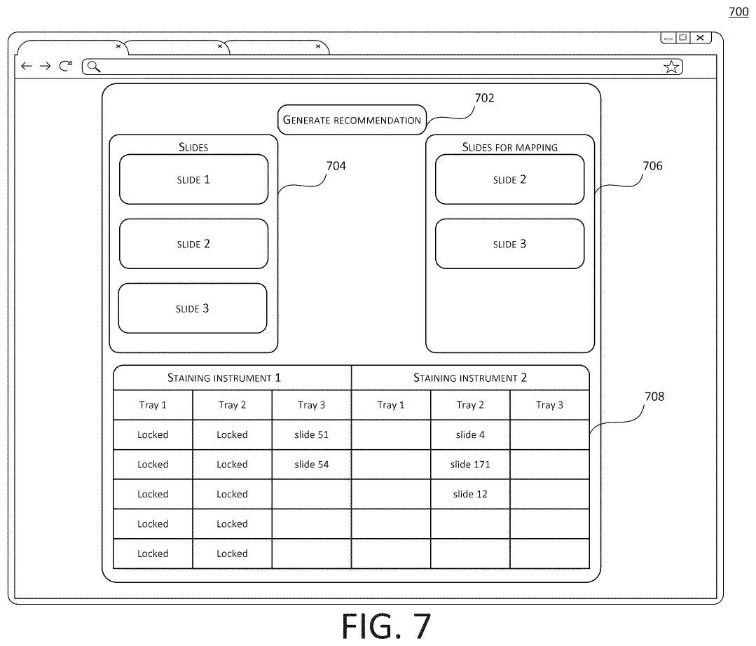

FIG. 7 depicts an example interface 700 for managing one or more slides and mapping the one or more slides to one or more staining instruments. The example interface 700 is illustrative of an interface that the slide management system, for example, as illustrated in FIG. 7, can generate or cause to be generated and presented to a user (e.g., a lab manager, etc.) via a computing device (e.g., a lab manager computing device) when interacting with the slide management system. The slide management system or another computing system may cause the example interface 700 to be presented to the user. In the example of FIG. 7, the interface 700 includes various areas that enable various functionality of the interface 700. For example, the interface 700 includes an area 702, an area 704, an area 706, and an area 708 to request a dynamic mapping of the slides to the staining instruments. It will be understood that the interface 700 can include more, less, or different areas. As will be described in more detail below, the slide management system may present the information associated with or describing the slides and/or the staining instruments based on determining the a user is authorized to request a dynamic mapping (e.g., the user is associated with an authorized role (e.g., a lab manager). Further, the slide management system may provide the information associated with or describing the slides and/or the staining instruments for review by the user. It will be understood that FIG. 7 is illustrative only, and a slide management system may offer any type of information (via the interface 700) about the slides or the staining instruments.

The interface 700 may further include area 704. The area 704 may identify slides that the slide management system has identified (e.g., recognized, scanned, etc.). For example, the area 704 may identify previously unprocessed slides (e.g., slides that have not been provided to and/or processed by a staining instrument). The area 704 may further identify slides that a user has uploaded to the slide management system. For example, the area 704 may identify slides that a user has uploaded by scanning, selecting, etc. the slides. In some embodiments, the area 704 may include identifying information about each slide. For example, the area 704 may include a source, an origination date, etc. of the slide. A user may interact with the area 704 in order to gather or learn about the slides. For example, the user may be able to parse, scan, scroll, etc. the area 704 in order to identify the slides and/or information associated with the slides. Further, the user may select one or more slides in the area 704. In the example of FIG. 7, the area 704 includes a plurality of slides including “Slide 1,” “Slide 2,” and “Slide 3.” It will be understood that the area 704 can include more, less, or different slides and/or details associated with the slides.

The interface 700 may further include area 706. The area 706 may identify slides that the slide management system has obtained (e.g., identified, recognized, etc.) for mapping to staining instruments. For example, the area 706 may identify slides (e.g., via slide identifiers) that a user has selected for dynamic mapping to one or more staining instruments (e.g., staining instruments that the slide management system has identified as available). Further, the area 706 may identify slides that a user has selected (e.g., dragged and dropped) from the area 704. In some embodiments, the slide management system may suggest particular slides for mapping (e.g., all slides, important slides, slides that have been pending for a particular period of time). Therefore, the area 706 can identify slides for mapping to staining instruments. In some embodiments, the area 706 may include identifying information about each slide for mapping to staining instruments. For example, the area 706 may include a source, an origination date, a previous mapping, etc. of the slide. In the example of FIG. 7, the area 706 includes a plurality of slides including “Slide 2” and “Slide 3.” In some embodiments, when a slide is selected from area 704 for placement in area 706 for mapping, the slide identifier corresponding to the slide may be removed from area 704 and added to area 706. It will be understood that the area 706 can include more, less, or different slides and/or details associated with the slides.

The interface 700 may further include area 708. The area 708 may identify data associated with a plurality of staining instruments. For example, the area 708 may store and/or summarize data associated with a plurality of available staining instruments. In some embodiments, the area 708 may store and/or summarize data associated with all staining instruments associated with a user, lab, etc. The area 708 may provide information about each staining instrument, each rack (e.g., tray) within each staining instrument, and/or each location within each rack. For example, the area 708 may identify available locations within each rack of each staining instrument. Each rack may include a defined number of locations (e.g., 10 locations). The area 708 may identify locations as available or unavailable. For example, the area 708 may identify locations of slides in the staining instruments. Further, the area 708 may identify certain locations as locked. The area 708 may also identify a dynamic mapping produced by the slide management system in response to a request by the user. The area 708 may identify these locations as locked based upon obtaining a user designation, determining the staining instrument and/or a rack is unavailable, etc. The area 708 may further include additional metadata associated with the staining instruments. In the example of FIG. 7, the area 708 includes metadata identifying staining instruments, trays of each staining instrument, and locations within each tray. It will be understood that the area 708 may identify further details about one or more of the staining instruments. The area 708 further identifies a first staining instrument “Staining Instrument 1” with the trays “Tray 1,” “Tray 2,” and “Tray 3.” Each tray of the first staining instrument includes six locations. Each location within the first staining instrument includes additional information. A first location of the tray “Tray 1” of the first staining instrument “Staining Instrument 1” is identified as “Locked,” a second location is identified as “Locked,” a third location is identified as “Locked,” a fourth location is identified as “Locked,” and a fifth location is identified as “Locked.” A first location of the tray “Tray 2” of the first staining instrument “Staining Instrument 1” is identified as “Locked,” a second location is identified as “Locked,” a third location is identified as “Locked,” a fourth location is identified as “Locked,” a fifth location is identified as “Locked,” and a sixth location is identified as “Locked.” A first location of the tray “Tray 3” of the first staining instrument “Staining Instrument 1” is identified as “slide 51,” a second location is identified as “slide 54,” a third location is identified as “<blank>,” a fourth location is identified as “<blank>,” a fifth location is identified as “<blank>,” and a sixth location is identified as “<blank>.” The area 708 further identifies a second staining instrument “Staining Instrument 2” with the trays “Tray 1,” “Tray 2,” and “Tray 3.” Each tray of the first staining instrument includes six locations. Each location within the second staining instrument includes additional information. A first location of the tray “Tray 1” of the second staining instrument “Staining Instrument 2” is identified as “<blank>,” a second location is identified as “<blank>,” a third location is identified as “<blank>,” a fourth location is identified as “<blank>,” a fifth location is identified as “<blank>,” and a sixth location is identified as “<blank>.” A first location of the tray “Tray 2” of the first staining instrument “Staining Instrument 1” is identified as “slide 4,” a second location is identified as “slide 171,” a third location is identified as “slide 12,” a fourth location is identified as “<blank>,” a fifth location is identified as “<blank>,” and a sixth location is identified as “<blank>.” A first location of the tray “Tray 3” of the second staining instrument “Staining Instrument 2” is identified as “<blank>,” a second location is identified as “<blank>,” a third location is identified as “<blank>,” a fourth location is identified as “<blank>,” a fifth location is identified as “<blank>,” and a sixth location is identified as “<blank>.” It will be understood that the area 708 may identify more, less, or different slides and/or staining instruments.

The interface 700 may further include area 702. The area 702 may allow a user to request the generation of a recommendation (e.g., a dynamic mapping). For example, the interface 700 may enable a user to request mapping of particular slides using the areas 704 and 706. Based on the user interacting with the area 702, the interface 700 may cause a prompt to be sent to the slide management system. The prompt may request for the slide management system to generate the dynamic mapping of slides (e.g., the slides designated in area 706) to staining instruments. In some embodiments, upon receiving a response from the slide management system, the area 702 (or another area of the interface 700 such as area 708) may identify the response (e.g., identify the mapping).

### Generating a Recommendation

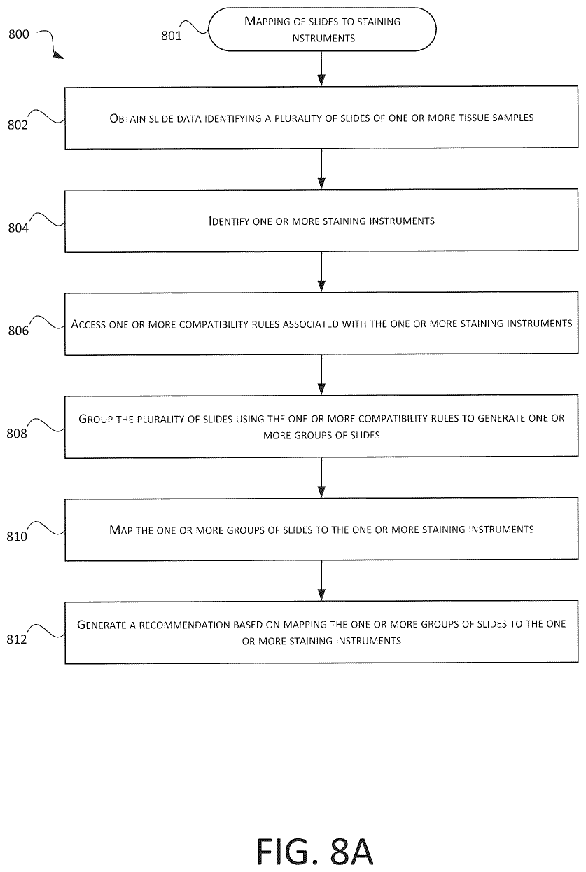

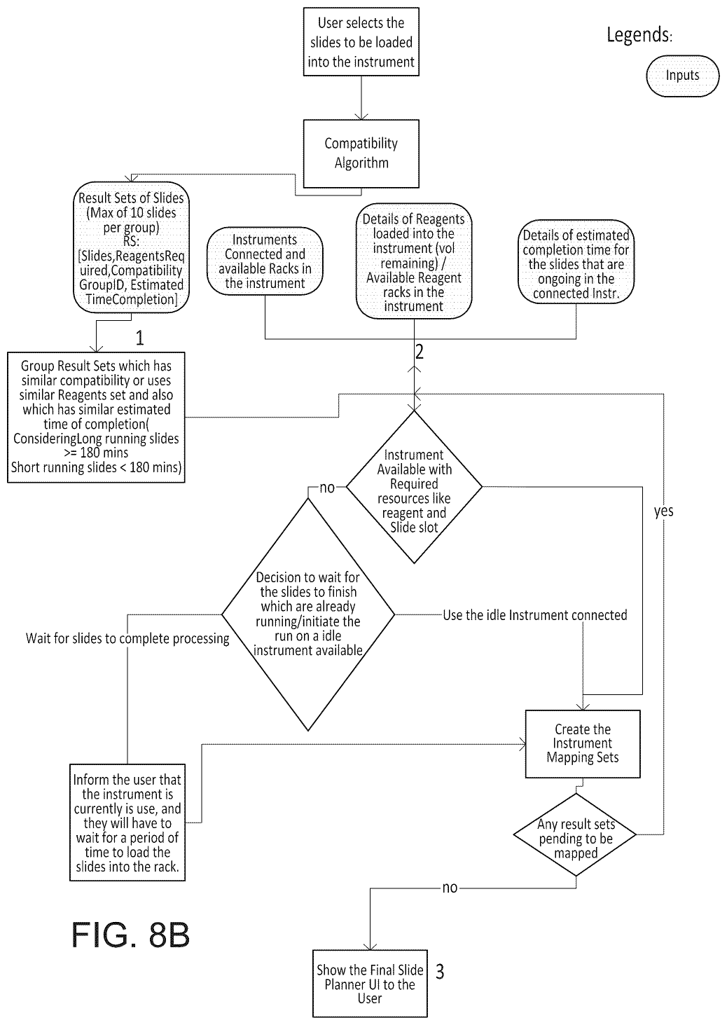

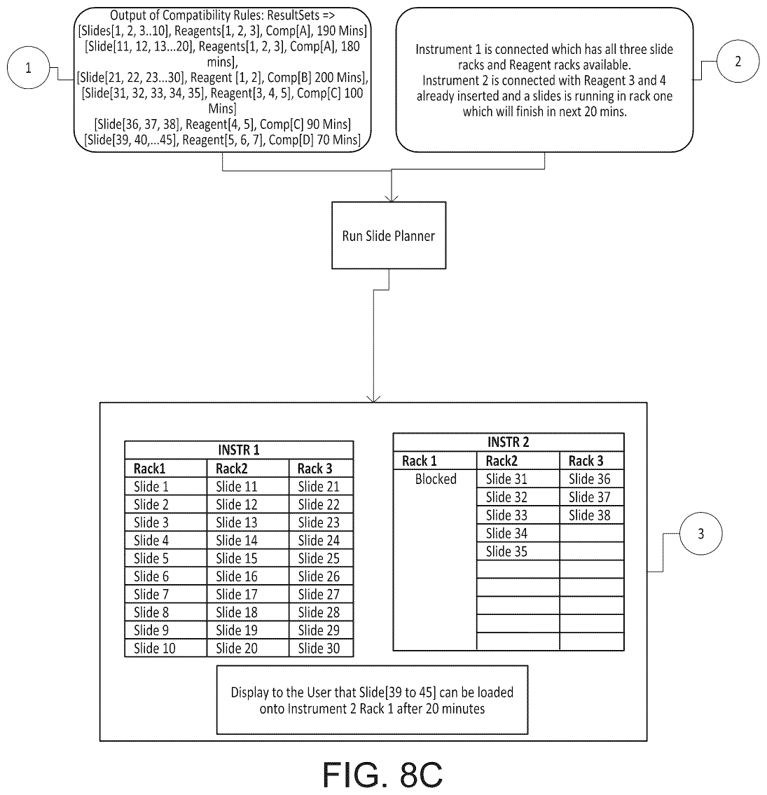

FIG. 8A shows a method 800 executed by an slide management system (e.g., a slide management apparatus), according to some examples of the disclosed technologies. The slide management system may be similar, for example, to the slide management system 104, and may include a microtome, a coverslipper, a staining instrument, one or more imaging devices, etc. Further, the slide management system may include a plurality of staining instruments that each stain slides of tissue samples to generate stained slides. It will be understood that the method 800 may be performed by different devices (e.g., a computing device). The process 800 may begin at block 801. The process 800 may begin automatically upon identifying slides.

In block 802, the slide management system obtains slide data identifying a plurality of slides of one or more tissue samples. The slide management system can obtain the slide data using a scanning device, a user computing device, or any other computing device. Prior to obtaining the slide data, the microtome may slice one or more tissue samples to generate the slides. A coverslipper may further be utilized to generate the slides. The tissue sample may further include a paraffin embedded tissue sample, an OCT-embedded tissue sample, a frozen tissue sample, or a fresh tissue sample. The slide management system may obtain the slide data based on scanning slide identifiers. The slide identifiers may be affixed to each slide and may include a tag, an RFID tag, a Bluetooth tag, an identifier, a barcode, a label, a marker, or a stamp.

In block 804, the slide management system identifies one or more staining instruments. The one or more staining instruments may include a first staining instrument and may identify available staining instruments. The one or more staining instruments may be available staining instruments, staining instruments with unoccupied racks or unoccupied positions, all staining instruments in a lab, etc. The slide management system may identify the one or more staining instruments based on obtained (e.g., identified) reagent availability or reagent rack availability for each staining instrument

In block 806, the slide management system accesses one or more compatibility rules associated with the one or more staining instruments. Each of the one or more compatibility rules may identify a characteristic and a corresponding characteristic value (e.g., a value or a range of values) of a corresponding staining instrument of the one or more staining instruments. The one or more compatibility rules can include one or more staining checks, one or more protocol checks, and one or more slide step checks. Further, the one or more compatibility rules can include a staining method, a staining mode, a slide coverage, a protocol count, a protocol type, a step count, a preferred kit, a fan speed, a fan duration, a dispense profile, a heat application order, or a mixed reagent.

In block 808, the slide management system groups the plurality of slides using the one or more compatibility rules to generate one or more groups of slides. Each of group of slides can include a group of compatible slides. For example, the group of compatible slides may be defined based on the compatibility rules. Each group of compatible slides may share a range of similarity. The range of similarity may be defined for each staining instrument of the one or more staining instruments. For example, each staining instrument may correspond to different ranges. In some embodiments, a given range of similarity may correspond to an exact (e.g., particular, single, etc.) value. Further, a particular staining instrument may correspond to a first range of similarity for a first characteristic (e.g., between 5 and 10 miles per hour for a fan speed characteristic) and a second range of similarity for a second characteristic (e.g., 20 steps for a step count characteristic). A first slide of a first group of slides may have a first characteristic value for a characteristic and a second slide of a second group of slides may have a second characteristic value for the characteristic (e.g., the first characteristic value and the second characteristic value may not be within a same range of similarity). Further, a first slide of a first group of slides may have a first characteristic value for a characteristic and a second slide of the first group of slides may have a second characteristic value different from the first characteristic value for the characteristic (e.g., the first characteristic value and the second characteristic value may be within a same range of similarity).

In block 810, the slide management system maps (e.g., dynamically maps) the one or more groups of slides to the one or more staining instruments. Mapping the groups of slides may include dynamically mapping a first group of slides to a first staining instrument, a second group of slides to a second staining instrument, etc. In dynamically mapping the slides to the staining instruments, the slide management system may confirm that the staining instruments have the necessary reagents for a particular group of slides. For example, if the slide management system determines that a staining instrument does not have the necessary reagent(s) for a particular group of slides, the slide management system may notify the user regarding which reagent(s) need to be loaded along with the slides. Further, the slide management system may determine a staining completion time for each of one or more groups of slides based on the staining instrument dynamically mapped to a particular group of slides and the dynamic mapping of the slides may be based on the staining completion time. For example, the dynamic mapping may be based on the most efficient (e.g., with respect to time) mapping of slides to staining instruments.

In block 812, the slide management system generates a recommendation based on dynamically mapping the one or more groups of slides to the one or more staining instruments. The recommendation may identifies racks of the staining instruments for placement of the groups of slides (e.g., particular racks of the first staining instrument for the first group of slides). In some embodiments, the recommendation may identify an optimized assignment for the slides. The slide management system can further cause a first staining instrument to stain a first group of slides in response to receiving a user acceptance of the recommendation. For example, in response to receiving the user acceptance, the slide management system can cause the slides to be moved (e.g., uploaded) to the first staining instrument. In some embodiments, the slide management system may receive confirmation that the slides have been moved to the first staining instrument (e.g., manually or via an automated process) and initiate the first staining instrument to stain the first group of slides. The slide management system may provide the recommendation via an application programming interface. Further, the slide management system can transmit the recommendation to a user computing device associated with a user (e.g., the slide management system can cause display of the recommendation via the user computing device). The slide management system may obtain a response to the recommendation and update the dynamic mapping of slides to staining instruments by dynamically mapping the first group of slides to a different staining instrument (e.g., a second staining instrument) based on the response.

In accordance with one or more related aspects, FIG. 8B provides a flow diagram for an example workflow utilizing the technique of method 800 described above with reference to FIG. 8A. FIG. 8C provides further information regarding certain features shown in FIG. 8B.

### Example Computing System

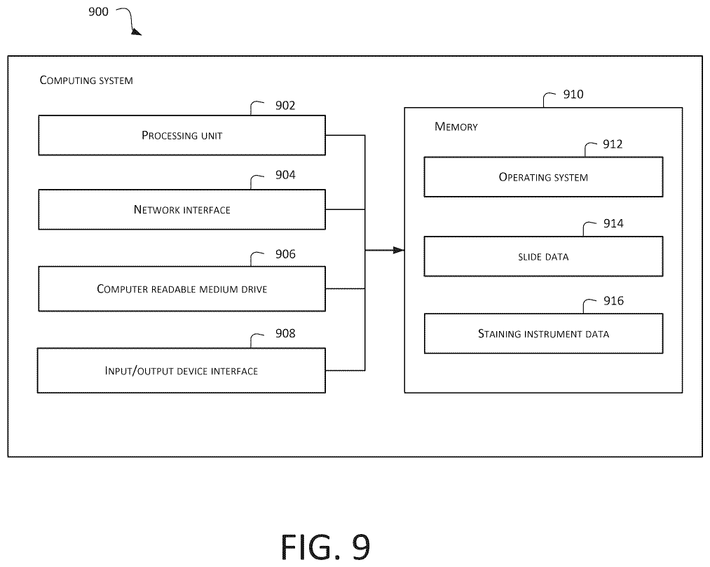

FIG. 9 illustrates an example computing system 900 configured to execute the processes and implement the features described above. In some embodiments, the computing system 900 may include: one or more computer processors 902, such as physical central processing units (“CPUs”); one or more network interfaces 904, such as a network interface cards (“NICs”); one or more computer readable medium drives 906, such as a high density disk (“HDDs”), solid state drives (“SDDs”), flash drives, and/or other persistent non-transitory computer-readable media; an input/output device interface 908, such as an input/output (“IO”) interface in communication with one or more microphones; and one or more computer readable memories 910, such as random access memory (“RAM”) and/or other volatile non-transitory computer-readable media.

The network interface 904 can provide connectivity to one or more networks or computing systems, e.g., transmitter(s), receiver(s), and/or transceiver(s). The computer processor 902 can receive information and instructions from other computing systems or services via the network interface 904. The network interface 904 can also store data directly to the computer-readable memory 910. The computer processor 902 can communicate to and from the computer-readable memory 910, execute instructions and process data in the computer readable memory 910, etc.

The computer readable memory 910 may include computer program instructions that the computer processor 902 executes in order to implement one or more embodiments. The computer readable memory 910 can store an operating system 912 that provides computer program instructions for use by the computer processor 902 in the general administration and operation of the computing system 900. The computer readable memory 910 can further include computer program instructions and other information for implementing aspects of the present disclosure. For example, in one embodiment, the computer readable memory 910 may include slide data 914. As another example, the computer-readable memory 910 may include staining instrument data 916. In some embodiments, multiple computing systems 900 may communicate with each other via respective network interfaces 904, and can implement multiple sessions each session with a corresponding connection parameter (e.g., each computing system 900 may execute one or more separate instances of the process 800), in parallel (e.g., each computing system 900 may execute a portion of a single instance of a process 800), etc.

### Certain Terminology

Terms of orientation used herein, such as “top,” “bottom,” “proximal,” “distal,” “longitudinal,” “lateral,” and “end,” are used in the context of the illustrated example. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular,” “cylindrical,” “semi-circular,” or “semi-cylindrical” or any related or similar terms, are not required to conform strictly to the mathematical definitions of circles or cylinders or other structures, but can encompass structures that are reasonably close approximations.

Conditional language, such as “can,” “could,” “might,” or “may,” unless specifically stated otherwise, or otherwise understood within the context as used, is generally intended to convey that certain examples include or do not include, certain features, elements, and/or steps. Thus, such conditional language is not generally intended to imply that features, elements, and/or steps are in any way required for one or more examples.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

The terms “approximately,” “about,” and “substantially” as used herein represent an amount close to the stated amount that still performs a desired function or achieves a desired result. For example, in some examples, as the context may dictate, the terms “approximately,” “about,” and “substantially,” may refer to an amount that is within less than or equal to 10% of the stated amount. The term “generally” as used herein represents a value, amount, or characteristic that predominantly includes or tends toward a particular value, amount, or characteristic. As an example, in certain examples, as the context may dictate, the term “generally parallel” can refer to something that departs from exactly parallel by less than or equal to 20 degrees. All ranges are inclusive of endpoints.

### SUMMARY

Several illustrative examples of managing slides of a tissue block sample have been disclosed. Although this disclosure has been described in terms of certain illustrative examples and uses, other examples and other uses, including examples and uses which do not provide all of the features and advantages set forth herein, are also within the scope of this disclosure. Components, elements, features, acts, or steps can be arranged or performed differently than described and components, elements, features, acts, or steps can be combined, merged, added, or left out in various examples. All possible combinations and subcombinations of elements and components described herein are intended to be included in this disclosure. No single feature or group of features is necessary or indispensable.

Certain features that are described in this disclosure in the context of separate implementations can also be implemented in combination in a single implementation. Conversely, various features that are described in the context of a single implementation also can be implemented in multiple implementations separately or in any suitable subcombination. Moreover, although features may be described above as acting in certain combinations, one or more features from a claimed combination can in some cases be excised from the combination, and the combination may be claimed as a subcombination or variation of a subcombination.

Any portion of any of the steps, processes, structures, and/or devices disclosed or illustrated in one example in this disclosure can be combined or used with (or instead of) any other portion of any of the steps, processes, structures, and/or devices disclosed or illustrated in a different example or flowchart. The examples described herein are not intended to be discrete and separate from each other. Combinations, variations, and some implementations of the disclosed features are within the scope of this disclosure.

While operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Additionally, the operations may be rearranged or reordered in some implementations. Also, the separation of various components in the implementations described above should not be understood as requiring such separation in all implementations, and it should be understood that the described components and systems can generally be integrated together in a single product or packaged into multiple products. Additionally, some implementations are within the scope of this disclosure.

Further, while illustrative examples have been described, any examples having equivalent elements, modifications, omissions, and/or combinations are also within the scope of this disclosure. Moreover, although certain aspects, advantages, and novel features are described herein, not necessarily all such advantages may be achieved in accordance with any particular example. For example, some examples within the scope of this disclosure achieve one advantage, or a group of advantages, as taught herein without necessarily achieving other advantages taught or suggested herein. Further, some examples may achieve different advantages than those taught or suggested herein.

Some examples have been described in connection with the accompanying drawings. The figures are drawn and/or shown to scale, but such scale should not be limiting, since dimensions and proportions other than what are shown are contemplated and are within the scope of the disclosed invention. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, any methods described herein may be practiced using any device suitable for performing the recited steps.

For purposes of summarizing the disclosure, certain aspects, advantages and features of the inventions have been described herein. Not all, or any such advantages are necessarily achieved in accordance with any particular example of the inventions disclosed herein. No aspects of this disclosure are essential or indispensable. In many examples, the devices, systems, and methods may be configured differently than illustrated in the figures or description herein. For example, various functionalities provided by the illustrated modules can be combined, rearranged, added, or deleted. In some implementations, additional or different processors or modules may perform some or all of the functionalities described with reference to the examples described and illustrated in the figures. Many implementation variations are possible. Any of the features, structures, steps, or processes disclosed in this specification can be included in any example.

In summary, various examples of managing the mapping of slides to staining instruments have been disclosed. This disclosure extends beyond the specifically disclosed examples to other alternative examples and/or other uses of the examples, as well as to certain modifications and equivalents thereof. Moreover, this disclosure expressly contemplates that various features and aspects of the disclosed examples can be combined with, or substituted for, one another. Accordingly, the scope of this disclosure should not be limited by the particular disclosed examples described above, but should be determined only by a fair reading of the claims. In some embodiments, the slide management systems disclosed herein can be used to manage slides of other samples different than a slide of a histological sample.

What is claimed is:

1. A slide management apparatus, comprising:
a transceiver; and
a computing device comprising a hardware processor configured to:
receive slide data, wherein the slide data identifies a plurality of slides of one or more tissue samples;
identify a first staining instrument from a plurality of staining instruments;
access one or more compatibility rules associated with the first staining instrument, wherein each of the one or more compatibility rules relates to a characteristic of the first staining instrument;
group the plurality of slides using the one or more compatibility rules to generate one or more groups of slides, wherein each of the one or more groups of slides comprises a group of compatible slides;
map a first group of slides of the one or more groups of slides to the first staining instrument; and
cause the transceiver to transmit information indicative of the mapping of the first group of slides to the first staining instrument.

2. The slide management apparatus of claim 1, wherein the hardware processor is further configured to cause the first staining instrument to stain the first group of slides in response to receiving a user acceptance of the information indicative of the mapping of the first group of slides to the first staining instrument.

3. The slide management apparatus of claim 1, wherein a first slide of the first group of slides of the one or more groups of slides is associated with a first characteristic value for a characteristic, wherein a second slide of a second group of slides of the one or more group of slides is associated with a second characteristic value for the characteristic, wherein the first characteristic value and the second characteristic value comprise different characteristic values.

4. The slide management apparatus of claim 1, wherein the one or more compatibility rules comprise:
a staining check;
a protocol check;
a slide step check;
a staining method;
a staining mode;
a slide coverage;
a protocol count;
a protocol type,
a step count;
a preferred kit;
a fan speed;
a fan duration;
a dispense profile;
a heat application order; or
a mixed reagent.

5. The slide management apparatus of claim 1, wherein the hardware processor is further configured to determine a staining completion time for each of the one or more groups of slides based on a staining instrument of the plurality of staining instruments mapped to a particular group of slides, wherein the hardware processor is configured to map the first group of slides to the first staining instrument based on the staining completion time.

6. The slide management apparatus of claim 1, wherein to identify the first staining instrument from the plurality of staining instruments, the hardware processor is further configured to:
receive one or more of a rack availability or a reagent rack availability for the first staining instrument; and
identify an available staining instrument of the plurality of staining instruments, wherein the available staining instrument comprises the first staining instrument.

7. The slide management apparatus of claim 1, wherein the information indicative of the mapping of the first group of slides to the first staining instrument identifies racks of the first staining instrument for placement of the first group of slides.

8. The slide management apparatus of claim 1, wherein the slide of the tissue sample is associated with at least one of:
a tag;
an RFID tag;
a Bluetooth tag;
an identifier;
a barcode;
a label;
a marker; or
a stamp.

9. The slide management apparatus of claim 1, wherein the hardware processor is further configured to:
cause the transceiver to transmit the information indicative of the mapping of the first group of slides to the first staining instrument to a user computing device associated with a user; and
cause display of the information indicative of the mapping of the first group of slides to the first staining instrument via the user computing device.

10. The slide management apparatus of claim 1, wherein the hardware processor is further configured to:
receive, via the transceiver, a response to the information indicative of the mapping of the first group of slides to the first staining instrument; and
update a mapping of the first group of slides to the first staining instrument, wherein to update the mapping of the first group of slides to the first staining instrument, the hardware processor is further configured to map the first group of slides of the one or more groups of slides to a second staining instrument of the plurality of staining instruments based on the response to the information indicative of the mapping of the first group of slides to the first staining instrument.

11. A computer-implemented method comprising:
receiving slide data, wherein the slide data identifies a plurality of slides of one or more tissue samples;
identifying a first staining instrument from a plurality of staining instruments, the first staining instrument configured to stain a slide of a tissue sample to generate a stained slide;
accessing one or more compatibility rules associated with the first staining instrument, wherein each of the one or more compatibility rules relates to a characteristic of the first staining instrument;
grouping the plurality of slides using the one or more compatibility rules to generate one or more groups of slides, wherein each of the one or more groups of slides comprises a group of compatible slides;
mapping a first group of slides of the one or more groups of slides to the first staining instrument; and
causing a transceiver to transmit information indicative of the mapping of the first group of slides to the first staining instrument.

12. A non-transitory computer-readable medium storing computer-executable instructions that, when executed by one or more computing devices of a slide management apparatus, cause the one or more computing devices to:
receive slide data, wherein the slide data identifies a plurality of slides of one or more tissue samples;
identify a first staining instrument from a plurality of staining instruments, the first staining instrument configured to stain a slide of a tissue sample to generate a stained slide;
access one or more compatibility rules associated with the first staining instrument, wherein each of the one or more compatibility rules relates to a characteristic of the first staining instrument;
group the plurality of slides using the one or more compatibility rules to generate one or more groups of slides, wherein each of the one or more groups of slides comprises a group of compatible slides;
map a first group of slides of the one or more groups of slides to the first staining instrument; and
cause a transceiver to transmit information indicative of the mapping of the first group of slides to the first staining instrument.

13. The non-transitory computer-readable medium of claim 12, wherein a first slide of the first group of slides of the one or more groups of slides is associated with a first characteristic value for a characteristic, wherein a second slide of a second group of slides of the one or more group of slides is associated with a second characteristic value for the characteristic, wherein the first characteristic value and the second characteristic value comprise different characteristic values.

14. The non-transitory computer-readable medium of claim 12, wherein the one or more compatibility rules comprise:
a staining check;
a protocol check;
a slide step check;
a staining method;
a staining mode;
a slide coverage;
a protocol count;
a protocol type,
a step count;
a preferred kit;
a fan speed;
a fan duration;
a dispense profile;
a heat application order; or
a mixed reagent.

15. The non-transitory computer-readable medium of claim 12, wherein the execution of the computer-executable instructions further causes the one or more computing devices to determine a staining completion time for each of the one or more groups of slides based on a staining instrument of the plurality of staining instruments mapped to a particular group of slides, wherein, to map the first group of slides to the first staining instrument, the execution of the computer-executable instructions further causes the one or more computing devices to map the first group of slides to the first staining instrument based on the staining completion time.

16. The non-transitory computer-readable medium of claim 12, wherein, to identify the first staining instrument from the plurality of staining instruments, the execution of the computer-executable instructions further causes the one or more computing devices to:
receive one or more of a rack availability or a reagent rack availability for the first staining instrument; and
identify an available staining instrument of the plurality of staining instruments, wherein the available staining instrument comprises the first staining instrument.

17. The non-transitory computer-readable medium of claim 12, wherein the information indicative of the mapping of the first group of slides to the first staining instrument identifies racks of the first staining instrument for placement of the first group of slides.

18. The non-transitory computer-readable medium of claim 12, wherein the slide of the tissue sample is associated with at least one of:
a tag;
an RFID tag;
a Bluetooth tag;
an identifier;
a barcode;
a label;
a marker; or
a stamp.

19. The non-transitory computer-readable medium of claim 12, wherein the execution of the computer-executable instructions further causes the one or more computing devices to:
cause the transceiver to transmit the information indicative of the mapping of the first group of slides to the first staining instrument to a user computing device associated with a user; and
cause display of the information indicative of the mapping of the first group of slides to the first staining instrument via the user computing device.

20. The non-transitory computer-readable medium of claim 12, wherein the execution of the computer-executable instructions further causes the one or more computing devices to:
receive, via the transceiver, a response to the information indicative of the mapping of the first group of slides to the first staining instrument; and
update a mapping of the first group of slides to the first staining instrument, wherein to update the mapping of the first group of slides to the first staining instrument, the execution of the computer-executable instructions further causes the one or more computing devices to map the first group of slides of the one or more groups of slides to a second staining instrument of the plurality of staining instruments based on the response to the information indicative of the mapping of the first group of slides to the first staining instrument.
